(19) United States
(12) Patent Application Publication
(10) Pub. No.: US 2005/0080310 A1
(43) Pub. Date: Apr. 14, 2005

(54) Method and device for separating hydrocarbon fluids

(76) Inventor: Kristian Utkilen, Bergen (NO)

Correspondence Address: Francis C Hand Carella Byrne Bain;Gilfillan Cecchi Stewart & Olstein, 5 Becker Farm Road, Roseland, NJ, 07068, US

(21) Appl. No.: 10/504,255

(22) PCT Filed: Feb. 18, 2003
(86) PCT No.: PCT/NO03/00062

(30) Foreign Application Priority Data
Feb. 18, 2002 (NO) 20020783

(51) Int. Cl. C07C 7/144

(52) U.S. Cl. 585/818; 585/800; 196/116

(57) ABSTRACT

A method for handling of hydrocarbon gas which is conducted to a flare for burning off is described. The method is characterised in that the hydrocarbon gas is separated into two or more fractions (parts), of which one fraction which has the desired quality is led to be burnt offin the flare, while the other fraction(s) is recovered. A device for carrying out the method is also described, and an application thereof.

The present invention relates to a method and a device for handling of hydrocarbon fluids (gases) that are meant to be led to a flare to be burnt off in connection with oil, gas and product installations, as described in the introduction in the subsequent claims 1 and 7, respectively.

The invention also relates to a particularly preferred application of the invention.

The aim of the invention is to reduce flaring and at the same time get a cleaner flame without smoke.

It is often a requirement that flares installed on oil and gas and product installations shall not have visible smoke. Smoke is due to incomplete combustion, and often occurs when heavier hydrocarbons are burnt off. At the same time, these hydrocarbons are as a rule the most valuable, and they can be sold on the market as clean products, as condensate or as a part of an oil product which is exported.

Hydrocarbons consist of a mixture of combustible molecules, i.e. everything from methane, ethane, propane and molecules with a higher molecular weight (i.e. with a higher number of carbon atoms in the molecule chain). The higher the average molecular weight (larger fraction of heavy components) a mixture has, the stronger the tendency for incomplete combustion and formation of soot particles. The “lighter” the mixture is (mainly such as ethane and methane), based on the mentioned molecular weight, the cleaner the flame and the less soot one gets from the combustion. A hydrocarbon mixture that contains a larger fraction of hydrocarbons, from propane and butane upwards, is regarded as a so-called “heavy” mixture.

A possible way to take care of such heavy components in the flare gas is to close the flare with respect to the methods that are described in API 521 (API—American Petroleum Institute).

One of these methods is to close the flare by means of a liquid seal. According to another of these methods, the flare is closed with the aid of valves and safety equipment in parallel. This method is described in Norwegian Patent Application 1993 1596 (NO Patent 177.161 (Statoil)) and implies that the surplus gasses are recovered. It is normally in emergency situations that the surplus gases are led to the flare to be burnt off.

The present invention relates to a further development in the form of an additional installation to the methods that are described in API 521 and as described in the abovementioned Norwegian patent.

It is an aim of the invention to provide a new method that can eliminate the disadvantages that exist in today's handling of gases which shall be flared.

Thus, it is an aim of the invention to remove the gas components which give the most polluting soot-containing, and thereby, black smoke discharges. These components are made up of the heavy hydrocarbons with a higher average molecular weight which result in that they do not burn completely or remain unburned, and thereby form soot particles and the associated pollution.

Furthermore it is an aim to be able to avoid the large investments, the extra flaring, and necessary shutdown of the site which otherwise will be necessary to avoid this soot-containing smoke.

It is a further aim of the invention to provide a method by which one can in a simple way retain the valuable components that would be able to give an economic contribution (an income) to the process.

It is yet another aim of the invention to provide a method by which one in a simple way can reduce the discharges of CO₂ and unburned components.

The method according to the invention is characterised by the features that are evident in the characteristics in the subsequent claim 1 and the associated dependent claims.

The device according to the invention is characterised by the features that are evident in the characteristic in the subsequent claim 7 and the associated dependent claims.

Further features of the inventive device are given in the dependent claims.

According to the invention the method and device to remove the “heavy” hydrocarbon components which form the soot when burnt from the “light” hydrocarbons that shall be burnt off in a flare, is applied as it appears in claim 13. The “heavy” hydrocarbon components that form the soot when burnt are preferably returned to the source process which is processing of crude oil as described in claim 14.

According to the invention a system can be installed in parallel, in which a fraction of the flare gas is recovered. Such a system can incorporate a membrane pack that separates the heavier hydrocarbons from the light hydrocarbons. The heavier hydrocarbons are retained while the light hydrocarbons are still burned. In this way one will obtain partial recovery of flare gases, with the most valuable components being recovered while the other components are flared.

With the invention one has thereby achieved that a larger fraction of the hydrocarbon gas (with a larger fraction of heavy components in the mixture) can be sent to recovery, while the remaining gas, which in the main consists of light hydrocarbons, can be burned off in the flare tower with a clean flame without smoke, or that the light components are discharged unburned in the flare (cold flare).

The separation element can be a membrane that separates the heavy hydrocarbons from the light hydrocarbons. A membrane normally needs pressure support from the feeding side and/or the aid of suction on the permeate side. The separation element will therefore often in addition to membranes encompass compressors upstream of the membranes or vacuum pumps downstream of the membranes.

Thus, the membrane functions by separating the original mixture into two fractions. One fraction gets a reduced overall molecular weight and burns with a cleaner flame, while the other fraction gets an increasing proportion of molecules with a higher molecular weight, and is thereby wanted returned to the process.

An example of a concrete embodiment or solution can be that the flare is closed, for example, by the aid of a liquid seal or a valve with safety equipment, in the form of pipes with valves in parallel, and, for example, comprising a safety valve (with rupture disc). In parallel to this, the membrane is installed which separates out the heavier components that are recovered, for example, with the aid of a compressor, and are led to a suitable location. The membrane is fitted in a chamber/housing-forming separation unit. Because of the back-pressure which the liquid seal (or safety equipment with valves) exerts against the fluid stream in the main pipe, the gas is led through the pipe to the chamber with the membrane. If too much gas flows through the pipe 10 so that the pressure increases, the gas will gradually begin to flow through the liquid seal (valve system) and thereby relieve the pressure increase.

This solution will be interesting for many installations. In particular installations that have no export possibilities for gas, but can handle components in liquid form. These installation will be able to retain the heavier components and lead them to recovery (for example back to the crude oil), and will with the aid of this solution obtain possibilities to increase the income as well as reduce polluting discharges.

Installations which have problems with smoke discharges will also be able to reduce, possibly completely eliminate, this problem by implementing this solution.

In the following the invention shall be explained further with reference to the enclosed figures, in which:

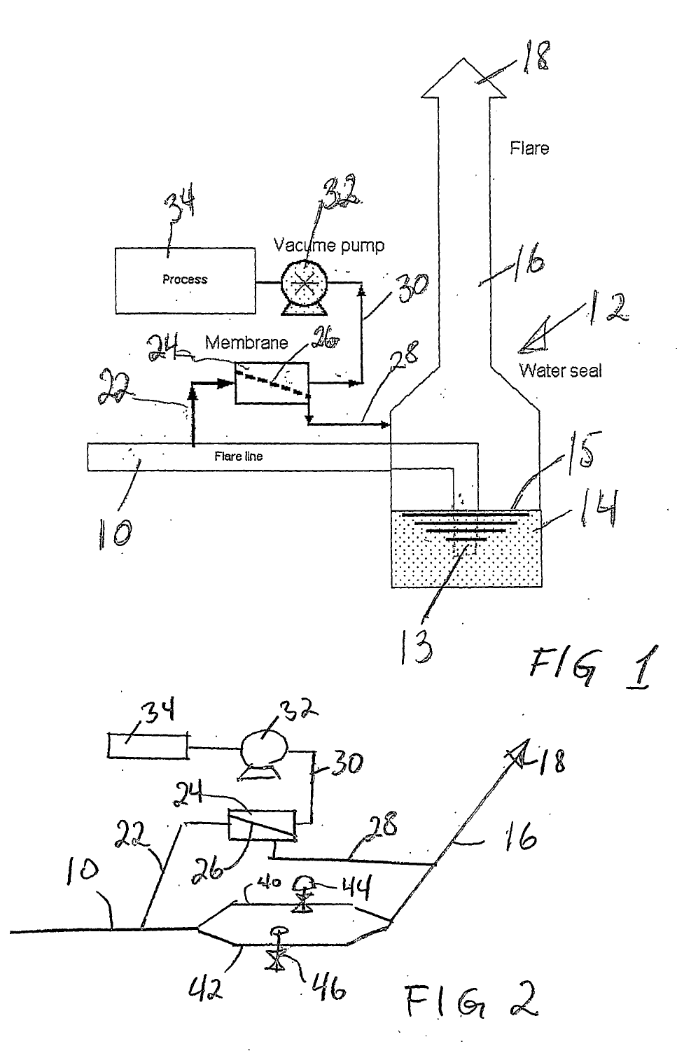

FIG. 1 shows an installation which handles a gas, such as surplus gases, for separation, according to the invention.

FIG. 2 shows the same separation system as in FIG. 1, but comprises an alternative solution for the safety system.

A flare gas comprised of hydrocarbons is conducted through a pipe 10 from a source, not shown, and to a liquid seal 12 that comprises a chamber 14 filled with liquid. The end 13 of the pipe 10 extends down into the liquid. A flare pipe 16 (a tube) protrudes from the upper part of the liquid seal 14 and leads the gas on to the flare 18 itself. The pressure which the gas in the pipe must have to rise further upwards in the flare tube, depends on how far the end 13 extends down into the liquid below the surface 15. Thus, the liquid seal construction provides safety if pressure increases arise in the tube 10. The gas will then be led out in the flare 18.

As a consequence of the pressure which the liquid seal in the flare tube 16 sets up, the gas is led through the pipe 22 to a chamber/housing-forming separation unit 24 that comprises a membrane 26. A membrane 26 is applied which will separate the gas into two fractions/flows of heavy and light hydrocarbon components, respectively. The light components are led through the pipe 28 and directly into the flare tube 16 to be burnt off in the flare 18. If too much gas comes through the pipe 10, so that the pressure increases, the gas will gradually begin to flow through the liquid seal (or the valve system) and thereby relieve the pressure increase.

From the other side of the membrane 26, a gas fraction of heavy components (i.e. the gas fraction that passes through the membrane) is led further through the pipe 30 to a compressor 32 where it is compressed and led into a process 34 for further treatment.

The further treatment can involve that the compressed fraction, together with crude oil, is conducted to further processing.

As the gas fraction of the light components which shall be burned off in the flare 18 now contains light components only, one obtains a clean flame without soot-forming, polluting, partly burnt hydrocarbons.

As an extra safety arrangement (or as a replacement for the liquid seal construction), a bypass pipe which can cut off the supply of gas to the separation unit 24 can be installed. The bypass pipe is initially closed with the aid of parallel-coupled safety valves, including an ordinary valve and a rupture-disc valve. The valve(s) in the pipe can be opened to lead gas directly to the flare 18 if faults arise in the separation unit 24, or unwanted pressure build-ups occur in the system, or other faults arise. The solution for the safety system with parallel pipes 40,42 between the inlet pipe 10 and flare tube 16, and with the installed valves 44,46 are outlined in FIG. 2. The valves 44,46 are set so that they open for flow to the flare tube 16 when the pressure is too high. One of these valves can include a safety-valve with a rupture disc which ruptures at a given higher pressure.

The separating component 26 is, as mentioned, a membrane which separates out the heavy hydrocarbons from the light hydrocarbons. The membrane 14 needs pressure-support on the feeding side, or the help of suction on the permeate side. Therefore, in addition to the membrane, one (or more) compressor(s) upstream of the membrane and/or vacuum pumps downstream of the membranes, are often installed. The membrane dissolves the heavier components and releases them on the other side of the membrane.

With the present invention, a solution is provided which will eliminate the problem of discharging solid soot particles from flares, something which constitutes a real pollution problem in addition to it being aesthetically unattractive.

The abovementioned example shall only be considered as an example, as the invention in its widest context is as described in the subsequent claims.

The mentioned solution will be interesting for many installations. In particular, installations which do not have possibilities for exporting gas but which can retain components in liquid form. These installations will get possibilities, by the aid of this solution, to increase the income as well as reduce discharges.

Installations which have problems with smoke discharges will also be able to reduce, possibly eliminate, this problem with the solution according to the invention.

1. Method for handling of hydrocarbon fluid (gases) which are led to a flare to be burnt off, characterised in that the hydrocarbon gas is separated into two or more fractions (parts), whereby one fraction which has the desired quality is led to be burnt off in the flare, while the other fraction(s) is recovered.

2. Method according to claim 1, characterised in that the hydrocarbon gas is separated by it being led through a membrane which separates light (hydrocarbon) components from heavy components, as the light components are led to the mentioned burning off.

3. Method according to claims 1-2, characterised in that the heavy hydrocarbon components are compressed and condensed to a liquid state and are mixed with a main product from the process which forms the combustible gases, said main product can be crude oil.

4. Method according to claims 1-3, characterised in that a safety system is applied in the form of a bypass of the fluid flow directly to the flare tube (16).

5. Method according to claims 1-4, characterised in that a pipe system with a liquid seal is used in the bypass.

6. Method according to claims 1-4, characterised in that for the bypass parallel pipes between the inlet pipe and flare tube are used, and with respective valves inserted, said valves open for flow to the flare tube at too high pressure, as a safety valve (with rupture disc) is used as one of the valves.

7. Device for handling of combustible hydrocarbon fluids (gases) which are intended to be burnt off in a flare, characterised by
means for separation of gases into two or more fractions (parts),
means for leading a fraction which has the desired quality to the combustion, and
means for transfer of the other fractions to recovery.

8. Device according to claim 7, characterised in that the separation means comprises a membrane which can separate light (hydrocarbon) components from heavy components.

9. Device according to claims 7-8, characterised in that a safety system comprising a bypass of the fluid stream directly to the flare tube.

10. Device according to claims 7-9, characterised in that the bypass comprises a pipe system with a liquid seal.

11. Device according to claims 7-10, characterised in that the bypass comprises parallel pipes between the inlet pipe and flare tube, and with inserted respective valves, said valves open for fluid flow to the flare tube when the pressure is too high, as a safety valve (with rupture disc) is used as one of the valves.

12. Device according to claims 7-11, characterised by compression equipment upstream or downstream of the membrane to obtain desired fractions of the hydrocarbons.

13. Application of method and device according to the preceding claims for the removal of the “heavy” hydrocarbon components that form soot when being burnt from the “light” hydrocarbons that shall be burnt off in a flare.

14. Application according to claim 7 for return of the “heavy” hydrocarbon components that form soot when being burnt to the source process which is processing of crude oil.
